(19) United States
(12) Patent Application Publication
(10) Pub. No.: US 2007/0292469 A1
(43) Pub. Date: Dec. 20, 2007

(54) Antimicrobial composition

(76) Inventors: Stephen J. Rothenburger, Neshanic Station, NJ (US); Xintian Ming, Bridgewater, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/188,444

(22) Filed: Jul. 25, 2005

(51) Int. Cl. A61K 31/785; A61K 33/38; A61F 2/02

(52) U.S. Cl. 424/423; 424/78.27; 424/618

(57) ABSTRACT

A stabilized antimicrobial composition comprising (a) polymeric polyquaternary ammonium compounds and (b) an antimicrobial metal, such as elemental silver, alloys thereof or silver compounds. The antimicrobial composition may be used as a stand-alone composition, in an antimicrobial formulation, or in combination with medical articles or medical devices. The antimicrobial composition prevents silver-containing formulations and medical devices from discoloration and loss of antimicrobial activity.

### FIELD OF INVENTION

This invention relates to a novel stabilized antimicrobial composition comprising (a) polymeric polyquaternary ammonium compounds and (b) one or more antimicrobial metal; and to methods of preparation and uses thereof. More specifically, this invention relates to a novel stabilized antimicrobial composition comprising polymeric polyquaternary ammonium compounds and elemental silver, alloys thereof or silver compounds. The present invention also relates to wound dressing, surgical dressing and other medical devices utilizing such novel stabilized antimicrobial compositions.

### BACKGROUND OF THE INVENTION

Each year, patients undergo a vast number of surgical procedures in the

United States. Current data shows about twenty-seven million procedures are performed per year. Post-operative or surgical site infections (“SSIs”) occur in approximately two to three percent of all cases. This amounts to more than 675,000 SSIs each year.

Whenever a medical device is used in a surgical setting, a risk of infection is created. The risk of infection dramatically increases for invasive or implantable medical devices, such as intravenous catheters, arterial grafts, intrathecal or intracerebral shunts and prosthetic devices, which create a portal of entry for pathogens while in intimate contact with body tissues and fluids. The occurrence of SSIs is often associated with bacteria that colonize on the medical device. For example, during a surgical procedure, bacteria from the surrounding atmosphere may enter the surgical site and attach to the medical device. Bacteria can use the implanted medical device as a pathway to surrounding tissue. Such bacterial colonization on the medical device may lead to infection and morbidity and mortality to the patient.

A number of methods for reducing the risk of infection associated with invasive or implantable medical devices have been developed that incorporate antimicrobial agents into the medical devices. Such devices desirably provide effective levels of antimicrobial agent while the device is being used. For example, medical devices may contain an antimicrobial agent such as silver.

The antimicrobial activity of silver compounds is a well known property which has been utilized for many years. More particularly, the antimicrobial effects are caused by silver ions that are released from, for example, silver compounds such as silver nitrate and silver sulfadiazine. Silver nitrate in concentrations of 0.5-1% (WN) in water shows disinfectant properties and is used for preventing infections in burns or for prophylaxis of neonatal conjunctivitis. Silver sulfadiazine is a silver complex, where both the sulfadiazine molecule and the silver ion have an antibacterial effect. Silver sulfadiazine is used intensively in the treatment of wounds, in particular for burns. Silver-protein-combinations are other antiseptic formulations which have been used in low concentrations, for example, in eye drops.

Antimicrobial agents based on silver compounds are also used in various medical devices. One example of such application is the use in the wound dressing sold by Johnson & Johnson under the trademark Actisorb®, which is an activated charcoal cloth dressing. Another example is the wound dressing sold under the trademark EZ-Derm by Genetic Laboratories, which is a modified pigskin impregnated with a soluble silver compound intended for treatment of burns. Additionally EP 272 149 B1 discloses a medical dressing of the “hydrocolloid” type containing, for example, silver chloride as an antiseptic compound.

A major drawback when using silver compounds that release silver ions for antimicrobial purposes is the dark stains that result on tissue or skin contacting the silver compound or the formulation or medical device having the silver compound incorporated thereon or therein. Such staining has been reported to give pigmentation of the skin, commonly referred to as argyria.

Although silver compounds are known to be efficacious antimicrobial agents, such compounds may also cause undesired changes in physical properties of formulations or medical devices having such silver compounds incorporated thereon or therein, both prior to and during use of the formulation or medical device. It is commonly recognized that formulations or medical devices containing silver compounds will discolor in the presence of an energy source, e.g. light and/or heat, or irradiation. For example, radiation sterilization can lead to an unsatisfactory change of color of a formulation such as a cream or a gel, or a medical device having a silver compound incorporated thereon or therein.

Photo-stable antimicrobial metal-based compositions have been disclosed in U.S. Pat. No. 6,468,521, in which the silver compound is described as a complex between the silver ions and a primary, secondary or tertiary lower alkyl amine or amino alcohol. This reference discloses that this complex is stable in the presence of hydrophilic polymers during sterilization and retains its antimicrobial activity, without giving rise to darkening or discoloration of the dressing during storage. However, it is undesirable to utilize lower alkyl amines or amino alcohols for medical applications, since lower alkyl amines or amino alcohols generally are known to be irritants and moderately toxic compounds having numerous potential side effects. Furthermore, the lower alkyl amine itself is not an effective antimicrobial agent.

One potential solution to the problem posed by the use of a lower alkyl amine described in U.S. Pat. No. 6,468,521, is the use of polymeric polyquaternary ammonium compounds and an antimicrobial metal to form a stabilized antimicrobial composition that is stable against discoloration upon exposure to light and/or heat and against the loss of antimicrobial activity.

Polymeric quaternary ammonium compounds are known to be less toxic and less irritating than monomeric amines. (Patty's Industrial Hygiene and Toxicology, Vol. II, Part B, 4th ed. 1994). An additional benefit of using polymeric polyquaternary ammonium compounds and an antimicrobial metal to form a stabilized antimicrobial composition is that polymeric quaternary ammonium compounds themselves are known to be effective antimicrobial agents. For instance, polyquaternium 1 demonstrates significant antimicrobial activity against a wide range of microorganisms, while lower alkyl amines such as monoethanolamines lack dramatic antimicrobial activity. (Disinfection, Sterilization, and Preservation 4th ed. 1991. S. Block ed. Lea & Febiger. pp 225, 232, 233, 313). Hence, the stabilized antimicrobial composition described herein possesses two active antimicrobial agent compared to the single antimicrobial agent described in U.S. Pat. No. 6,468,521.

There have been no reports to date on the use of a combination of (a) polymeric polyquaternary ammonium compounds, and (b) an antimicrobial metal, which has been discovered to be stable upon exposure to light and/or heat, while possessing enhanced antimicrobial activity. More specifically, the use of the stabilized antimicrobial composition described herein alone, in a formulation, or in conjunction with a medical device, prevents the formation of dark colored sparingly soluble or insoluble silver compounds (e.g. silver oxides) and the resultant discoloration of the formulation or medical device having the antimicrobial composition incorporated thereon or therein.

### SUMMARY OF THE INVENTION

Described herein is a stabilized antimicrobial composition comprising (a) polymeric polyquaternary ammonium compounds, and (b) one or more antimicrobial metal, such as elemental silver, alloys thereof or silver compounds. The antimicrobial composition may be used as a stand-alone antimicrobial composition, in a formulation such as a cream or a gel, or in combination with medical articles or medical devices.

### DETAILED DESCRIPTION OF THE INVENTION

The present invention is directed to an antimicrobial composition comprising (a) polymeric polyquaternary ammonium compounds and (b) one or more antimicrobial metal. More specifically, the present invention is directed to an antimicrobial composition comprising polymeric polyquaternary ammonium compounds and elemental silver, alloys thereof or silver compounds, that may be used alone, in a formulation or in combination with medical devices to impart antimicrobial properties to the formulation or device.

The polymeric polyquaternary ammonium compounds described herein have the following formula (I): 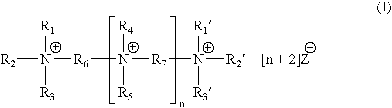
wherein R₁, R₂, R₃, R_{1′}, R_{2′}, R_{3′}, R₄, R₅, in the above formula may be identical or different, and are independently selected from hydrogen, an C₁-C₂₀ alkyl group, an aryl group, a benzyl group, an aralkyl group, or an alkylaryl group. Each C₁-C₂₀alkyl group may be substituted or un-substituted, linear or branched. R₆, and R₇ in the above formula may be identical or different, and are independently selected from (CH₂)ₘ, or (CH₂)ₘ —(CH═CH)_{m′}—(CH₂)ₘ, wherein 12=>m>=1, 10=>m′>=1 and 150>=n=>5; and Z is anionic moiety including without limitation, F, Cl, Br, I and COOH.

The polyquaternium polymer compounds suitably have a weight average molecular weight M_{w} most preferably about 4600 to 11,000.

A particular example of such class of polyquaternium polymer compound is polyquaternium-1: α-4-[1-tris(2-hydroxyethyl) ammonium-2-butenyl] poly[1-dimethylammonium-2-butenyl]-ω-tris(2-hydroxyethyl)ammonium chloride (available under the trademark Onamer M® from Onyx Chemical Company, Jersey City, N.J.; also known as Polyquad®, a registered trademark of Alcon Laboratories, Inc., Ft. Worth, Tex.; also known as polyquaternium-1), having the chemical structure described in formula (II): 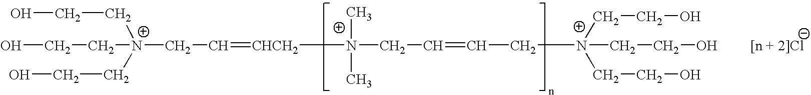
and may be made as the reaction product of 1,4-bis[dimethylamino]-2-butene (0.9 mol), triethanolamine (0.2 mol), and 1,4-dichloro-2-butene (1.0 mol) in water.

The polyquaternium polymer compounds may be in the form of a liquid concentrate, a salt, or a salt in aqueous solution. One particularly useful form of the polyquaternium polymer compounds is polyquaternium-1 chloride in aqueous solution.

The antimicrobial metal referred to herein is a metal having antimicrobial efficacy, including but not limited to Ag, Au, Pt, Pd, Ir, Sn, Cu, Sb, Bi, and Zn. The forms of the antimicrobial metal include, but are not limited to elemental, ionic, compounds, alloys or mixtures thereof.

Antimicrobial metals, in particular elemental silver, silver compounds, silver alloys or mixtures thereof, are especially potent against a broad spectrum of microorganisms. Preferably, the antimicrobial metal is elemental silver, silver compounds, alloys or mixtures thereof. The silver compound referred to herein is a compound comprising a silver ion, linked to another molecule via a covalent or non-covalent linkage with the potential to be oxidized to form silver oxide. An example of a silver compound includes, but is not limited to, silver salts formed by silver ions with organic acids (e.g. acetic acids and fatty acids) or inorganic acids, such as silver sulfadiazine (“AgSD”), silver carbonate (“Ag₂CO₃”), silver deoxycholate, silver salicylate, silver iodide, silver nitrate (“AgNO₃”), silver paraminobenzoate, silver paraminosalicylate, silver acetylsalicylate, silver ethylenediaminetetraacetic acid (“Ag EDTA”), silver picrate, silver protein, silver citrate, silver lactate, silver acetate and silver laurate.

In one particular set of non-limiting embodiments, the present invention provides an antimicrobial composition comprising a complex of polymeric polyquaternary ammonium compounds with one or more antimicrobial metal. The term “complex” as used herein refers to an intimate mixture at the molecular scale, preferably with ionic or covalent bonding between the antimicrobial metal and the polymeric polyquaternary ammonium compounds. The complex preferably comprises a salt formed between the polymeric polyquaternary ammonium compounds and ions of the antimicrobial metal, but it may also comprise metal clusters and/or colloidal metal, for example produced by exposure of the complex to light.

In one embodiment, the antimicrobial composition is in the form of an aqueous or organic solution of the polymeric polyquaternanry ammonium compound and an antimicrobial metal, which may be utilized in medical applications directly on tissue and skin, i.e., for the treatment of burns.

In another embodiment, the stabilized antimicrobial composition comprises (a) polymeric polyquaternary ammonium compounds (b) an antimicrobial metal, which may be incorporated into a formulation, independent of any medical devices or specific applications. Formulations of the antimicrobial composition may be of liquid (e.g. solutions) or solid form (e.g. powders), and may comprise the polymeric polyquaternary ammonium compound in an amount from about 0.001% to about 5% by weight and the antimicrobial metal in an amount from about 0.001% to about 5% by weight relative to total weight of the formulation. More preferably, the formulation may comprise the polymeric polyquaternary ammonium compound in an amount from about 0.01% to about 1% by weight and the antimicrobial metal in an amount from about 0.01% to about 1% by weight relative to total weight of the formulation. For instance, formulations having the antimicrobial composition may be in form of cream or gel and may be applied directly to a wound.

In another set of non-limiting embodiments, the antimicrobial composition may be incorporated on or into medical devices. The terms “incorporate”, “incorporated”, or “incorporating”, as used herein, refer to combining the composition with the medical device by physical or chemical means. Examples include, but are not limited to, impregnating, dipping, soaking or coating a medical device with an aqueous or organic solution of the antimicrobial composition or preparing the medical device by adding the antimicrobial composition to the material that the medical device is made from. The medical devices that may be treated are either fabricated from or coated or treated with a biomedical polymer and include, but are not limited to, microcapsules, dressings, implants, wound closures, staples, meshes, controlled drug delivery systems, wound coverings, fillers, sutures, tissue adhesives, tissue sealants, absorbable and non-absorbable hemostats, catheters including urinary catheters and vascular catheters (e.g., peripheral and central vascular catheters), wound drainage tubes, arterial grafts, soft tissue patches (such as polytetrafluoroethylene (“PTFE”) soft tissue patches), gloves, shunts, stents, tracheal catheters, wound dressings, sutures, guide wires and prosthetic devices (e.g., heart valves and LVADs). The present invention may be further applied to medical articles that have been prepared according to U.S. Pat. Nos. 3,839,297; 4,027,676; 4,185,637 and 4,201,216, the contents of which is hereby incorporated by reference herein as if set forth in its entirety.

The medical dressings that may be treated are protein or polysaccharide based. Polysaccharide is selected from the group consisting of cellulose derivatives, chitin, chitosans, galactomannans, alginate and mixtures thereof. Protein is selected from the group consisting of collagen, gelatin and mixture thereof. Examples of such polysaccharide based dressings commercially available include Surgicel® absorbable hemostat; Surgicel Nu-Knit® absorbable hemostat; and Surgicel® Fibrillar absorbable hemostat; protein based dressings include Surgifoam® absorbable gelatin; and Promogran® dressing having collagen and oxidized regenerated cellulose, all available from Johnson & Johnson Wound Management Worldwide, a division of Ethicon, Inc., Somerville, N.J., a Johnson & Johnson Company. Where the medical article is a dressing, such as Surgicele absorbable hemostat, a knitted fabric of oxidized regenerated cellulose (ORC), the amount of polymeric polyquaternary ammonium compound on the dressing may be from about 0.001-500 μg/cm², preferably from about 0.01-100 μg/cm², and the amount of silver metal may be from about 0.001-500 μg/cm², preferably 0.01-100 μg/cm². The term “about” as used herein indicates a variation within 20 percent.

The antimicrobial composition described herein is characterized by its stabilization effect to silver ions, thereby resulting in the prevention of discoloration of tissue or skin to which it is applied, or the formulation or medical device in which it is incorporated on or within. The use of this stabilized composition has also been shown to be effective against a broader antimicrobial spectrum of organisms including, but not limited, Tinea pedis, Tinea unguium, Tinea cruris, or Tinea capitis, S. aureus, MRSA, MRSE, GISA, S. epidermidis, E. coli, P. aeruginosa, K. pneumoniae, B. cepacia, E. cloacae, S. marcescens, S. pyogenes, S. agalacticae, E. faecalis-Vancomycin Resistant, E. faecium, C. albicans and B. subtilis, Salmonella sp., Proteus sp., Acinetobacter sp. Aspergillus niger.

While the following examples demonstrate certain embodiments of the invention, they are not to be interpreted as limiting the scope of the invention, but rather as contributing to a complete description of the invention.

### EXAMPLE 1

### Preparation of Stabilized Silver Compound

Stock solutions of Ag NO₃ at 0.1% and polyquaternium 1 (Onamer M, Stepan company, Northfield, Ill. USA) at 0.1% were prepared in distilled water respectively. The silver-polyquat (Ag NO₃— polyquaternium 1) complex was formed by mixing the two stock solutions at 1:1 ratio and was equilibrated for 24 hours at ambient temperature in the dark. The silver-polyquat complex solution was applied onto a white cellulose disc (1 cm diameter) at 100 ul/disc. The treated discs contained 100 ug silver nitrate (in the polyquat complex)/disc and was tested for light and heat stability as described in Example 5.

### EXAMPLE 2

The silver-polyquat complex was applied to Surgicel® absorbable hemostat, a knitted fabric of oxidized regenerated cellulose (Ethicon Inc, a Johnson and Johnson company, Somerville N.J., USA). The Surgicel® absorbable hemostat was cut into 1×1 cm squares. The silver-polyquat complex solution prepared in Example 1 was applied onto the Surgicel® absorbable hemostat at 50 ul/cm². Each Surgicel® absorbable hemostat section contained 50 ug silver nitrate (in the polyquat complex)/cm² and was tested for light stability in Example 5.

### EXAMPLE 3

The silver-polyquat complex was applied to Promogran® dressing, a collagen and oxidised regenerated cellulose product (Johnson & Johnson medical, Divison of Ethicon, Inc. Gargrave, U.K.). The Promogran® dressing was cut into 1×1 cm squares. The silver-polyquat complex solution prepared in Example 1 was applied onto the Promogran® dressing at 100 ul/cm². Each Promogran® dressing section contained 100 ug silver nitrate (in the polyquat complex)/cm² and was tested for light stability in Example 5.

### EXAMPLE 4

The silver-polyquat complex was applied to Nu-Gel® hydrogel (Johnson & Johnson medical, Divison of Ethicon, Inc. Gargrave, U.K.). The Nu-Gel® hydrogel was cut into 1×1 cm squares. The silver-polyquat complex solution prepared in Example 1 was applied onto the Nu-Gel® hydrogel section at 50 ul/cm². Each Nu-Gel® section contained 50 ug silver nitrate (in the polyquat complex)/cm² and was tested for light stability in Example 5.

### EXAMPLE 5

Comparison of the stabilized silver compositions on devices with silver alone on devices against discoloration by heat and light.

Stability against light was determined by exposing samples from Examples 1-4 to sunlight through a glass window for 1 to 6 hours. Discoloration was determined by visual observation.

Stability against heat was determined for samples from Examples 1-4, which were each sealed into autoclavable bags and subjected to heat treatment at 121° C. for 60 minutes.

Control samples (“Silver Alone” in Table 1) were prepared to contain the same amount of silver nitrate as the amount of silver nitrate in the polyquat complex, in each of the samples prepared in Example 1-4. The control samples were evaluated for stability against light and heat in the manner described above.

The use of the stabilized silver-polyquat complex prevented discoloration as illustrated by the results shown in Table 1. The control samples that contained 50 or 100 ug Ag NO₃ became dark brown after one hour exposure to sunlight, while the samples of Example 1-4 containing 50 ug or 100 ug silver-polyquat complex showed no color change after more than 6 hours exposure of sunlight.

TABLE 1
Silver stabilization against discoloration
by using Polyquaternium 1 (Onamer M)

| | Sunlight | | Heat | |
|---|---|---|---|---|
| | | Stabilized | | Stabilized |
| Example | Silver Alone | silver | Silver Alone | silver |
| 1 | dark brown | white | brown | white |
| 2 | yellow brown | white | N/A | N/A |
| 3 | dark brown | white | N/A | N/A |
| 4 | dark brown | white | N/A | N/A |

### EXAMPLE 6

Comparison of the antimicrobial efficacy of stabilized silver compositions on samples with samples having silver alone, after heat treatment and sunlight exposure

The sample of Example 1 that was tested in Example 5 was further evaluated for its antimicrobial efficacy. The samples were placed into Tryptic Soy agar medium inoculated with about 10⁵ cfu bacteria. Zones of inhibition were recorded after the plates were incubated at 37° C. for 24 hr. Zone of inhibition was defined as the distance from edge of the sample to the clear edge of bacterial lawn.

The results in Table 2 indicate that after exposure to sunlight or heat, the articles treated with the stabilized silver composition preserved theantimicrobial efficacy, while the control samples treated with silvers alone showed reduced efficacy by the same exposure. This effect was shown by the result of zone of inhibition against E. coli and E. faecium. No zone of inhibition against E. faecium was observed on samples containing AgNO₃ alone upon sunlight or heat exposure, which indicates a significant loss in efficacy by sunlight and heat exposure. The samples containing the stabilized silver composition showed no efficacy loss by the same exposure, indicated by similar zones of inhibition before and after the exposure.

TABLE 2

| | | Zone of inhibition (mm) | |
|---|---|---|---|
| Exposure | Discoloration | E. coli | E. faecium |

Without exposure

| | | | |
|---|---|---|---|
| 100 ug AgNO₃ on disc | white | 3.2 | 3.0 |
| (Prepared as “Silver | | | |
| Alone” in Example 5) | | | |
| 100 ug Polyquat on disc | white | 0 | 0 |
| 100 ug AgNO₃ on disc | white | 3.8 | 3.6 |
| (in the silver poylquat | | | |
| complex and prepared in | | | |
| Example 1) | | | |

Sunlight

| | | | |
|---|---|---|---|
| 100 ug AgNO₃ on disc | dark brown | 2.1 | 0 |
| (Prepared as “Silver | | | |
| Alone” in Example 5) | | | |
| 100 ug Polyquat on disc | white | 0 | 0 |
| 100 ug AgNO₃ on disc | white | 3.7 | 3.4 |
| (in the silver poylquat | | | |
| complex and prepared in | | | |
| Example 1) | | | |

Heat

| | | | |
|---|---|---|---|
| 100 ug AgNO₃ on disc | dark brown | unclear zone | 0 |
| (Prepared as “Silver | | | |
| Alone” in Example 5) | | | |
| 100 ug Polyquat on disc | white | 0 | 0 |
| 100 ug AgNO₃ on disc | white | 2.8 | 2.2 |
| (in the silver poylquat | | | |
| complex and prepared in | | | |
| Example 1) | | | |

### EXAMPLE 7

Comparison of stabilized silver-polyquat with stabilized silver-monomeric amines against discoloration by light.

Stock solutions of AgNO₃ at 0.1%, tri-hydroxymethyl-aminomethane (Tris, Sigma) at 0.1%, and polyquaternium 1 (Onamer M, Stepan company, Northfield, Ill. USA) at 0.1% were prepared in distilled water respectively. The silver-stabilized complexes were formed by mixing the silver nitrate stock solutions with either the polyquaternium 1 stock solution or the tri-hydroxymethyl-aminomethane stock solution at 1:1 ratio and were equilibrated for 24 hours at ambient temperature in the dark. The silver-stabilized complex solutions with either polyquaternium 1 or tri-hydroxymethyl-aminomethane were applied onto white cellulose disc (1 cm diameter) at 100 ul/disc. Silver nitrate alone was applied onto a separate white cellulose disc (1 cm diameter) at 100 ul/disc. All discs were exposed to sunlight for 1 hr and the discoloration was observed and recorded.

TABLE 3

| Sample | discoloration by sunlight exposure |
|---|---|
| 100 ug AgNO₃ on disc | dark brown |
| (Prepared as “Silver | |
| Alone” in Example 5) | |
| 100 ug AgNO₃ on disc | light brown |
| (in the silver Tris complex | |
| and prepared in Example 1) | |
| 100 ug AgNO₃ on disc | white |
| (in the silver poylquat | |
| complex and prepared in | |
| Example 1) | |

What is claimed is:

1. A stabilized antimicrobial composition comprising:
a polymeric polyquaternary ammonium compound, according to the following formula: 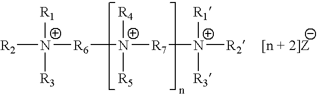
wherein R₁, R₂, R₃, R_{1′}, R_{2′}, R_{3′}, R₄, R₅, in the above formula may be identical or different, and are independently selected from hydrogen, an C₁-C₂₀ alkyl group, an aryl group, a benzyl group, an aralkyl group, or an alkylaryl group, and each C₁-C₂₀ alkyl group may be substituted or un-substituted, linear or branched; R₆, and R₇ in the above formula may be identical or different, and are independently selected from the group consisting of (CH₂)ₘ, and (CH₂)ₘ—(CH═CH)_{m′}—(CH₂)ₘ;
wherein 12=>m>=1, 10=>m′>=1 and 150>=n=>5; and Z is anionic moiety selected from the group consisting of F, Cl, Br, I and COOH;
and at least one antimicrobial metal.

2. The stabilized antimicrobial composition of claim 1, wherein the polymeric polyquaternary ammonium compound is polyquaternium-1, according to the following formula:

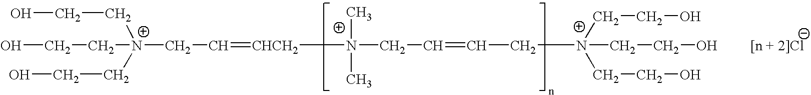

3. The stabilized antimicrobial composition of claim 1, wherein the antimicrobial metal is selected from the group consisting of elemental, ionic compounds, alloys or mixtures thereof.

4. The stabilized antimicrobial composition of claim 1, wherein the antimicrobial metal is selected from the group consisting of Ag, Au, Pt, Pd, Ir, Sn, Cu, Sb, Bi, and Zn.

5. The stabilized antimicrobial composition of claim 1, wherein the antimicrobial metal is silver.

6. A stabilized antimicrobial composition comprising:
polyquaternium-1 according to the following formula: 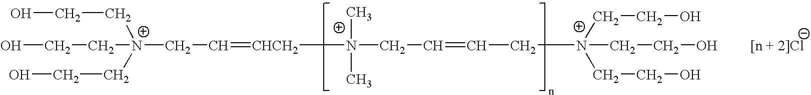
and silver or a silver compound.

7. A stabilized formulation or medical device comprising an antimicrobial composition that comprises:
a polymeric polyquaternary ammonium compound, according to the following formula: 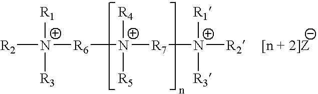
wherein R₁, R₂, R₃, R_{1′}, R_{2′}, R_{3′}, R₄, R₅, in the above formula may be identical or different, and are independently selected from hydrogen, an C₁-C₂₀ alkyl group, an aryl group, a benzyl group, an aralkyl group, or an alkylaryl group, and each C₁-C₂₀ alkyl group may be substituted or un-substituted, linear or branched; R₆, and R₇ in the above formula may be identical or different, and are independently selected from the group consisting of (CH₂)ₘ, and (CH₂)ₘ—(CH═CH)_{m′}—(CH₂)ₘ;
wherein 12=>m>=1, 10=>m′>=1 and 150>=n=>5; and Z is anionic moiety selected from the group consisting of F, Cl, Br, I and COOH;
and at least one antimicrobial metal.

8. The stabilized formulation or medical device of claim 7, wherein the polymeric polyquaternary ammonium compound is polyquaternium-1, according to the following formula:

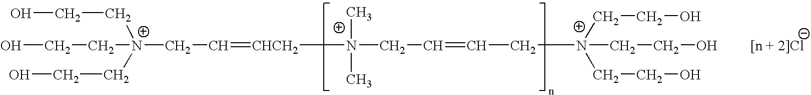

9. The stabilized formulation or medical device of claim 7, wherein the antimicrobial metal is selected from the group consisting of elemental, ionic compounds, alloys or mixtures thereof.

10. The stabilized formulation or medical device of claim 7, wherein the antimicrobial metal is selected from the group consisting of Ag, Au, Pt, Pd, Ir, Sn, Cu, Sb, Bi, and Zn.

11. The stabilized formulation or medical device of claim 7, wherein the antimicrobial metal is silver.

12. A stabilized formulation or medical device comprising:
polyquaternium-1 according to the following formula: 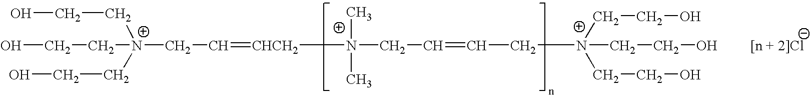
and silver or a silver compound.

13. The stabilized formulation or medical device of claim 12, wherein the concentration of said polyquaternium-1 is between about 0.001% and about 5% by weight based on the total weight, and of said silver compound is between about 0.001% and about 5% by weight based on the total weight of the stabilized formulation or medical device.

14. The stabilized formulation or medical device of claim 12, wherein the concentration of said polyquaternium-1 is between about 0.01% and about 1% by weight based on the total weight, and of silver compound is between about 0.01% and about 1% by weight based on the total weight of the stabilized formulation or medical device.
